(19) United States
(12) Patent Application Publication
(10) Pub. No.: US 2020/0254240 A1
(43) Pub. Date: Aug. 13, 2020

(54) Devices and Methods For The Incorporation Of A Microneedle Array Analyte-Selective Sensor Into An Infusion Set, Patch Pump, Or Automated Therapeutic Delivery System

(71) Applicant: Biolinq, Inc., San Diego, CA (US)

(72) Inventors: Joshua Windmiller, Del Mar, CA (US); Jared Rylan Tangney, Encinitas, CA (US); Thomas Arnold Peyser, Menlo Park, CA (US)

(73) Assignee: Biolinq, Inc., San Diego, CA (US)

(21) Appl. No.: 16/824,700

(22) Filed: Mar. 20, 2020

Related U.S. Application Data
(63) Continuation-in-part of application No. 16/666,259, filed on Oct. 28, 2019.
Application No. 16/666,259 is a continuation of application No. 16/152,372, filed on Oct. 4, 2018, now Pat. No. 10,492,708.
Application No. 16/152,372 is a continuation of application No. 15/590,105, filed on May 9, 2017, now Pat. No. 10,092,207.
(60) Provisional application No. 62/823,628, filed on Mar. 25, 2019.
(60) Provisional application No. 62/336,724, filed on May 15, 2016.

(51) Int. Cl. A61N 1/30; A61M 5/172; A61B 5/145; A61B 5/05; A61B 5/1468; A61N 1/05

(52) U.S. Cl. CPC A61N 1/30; A61M 5/1723; A61B 5/14546; Y02E 60/527; A61B 5/1468; A61N 1/05; A61B 2562/125; A61B 5/05

(57) ABSTRACT

A device and method for the manual delivery of a therapeutic intervention in response to a physiological state of a user is disclosed herein. The device comprises a sensor, an infusion system, and a singular body-worn component. The sensor is configured to penetrate the stratum corneum to access the viable epidermis or dermis and measure the presence of an analyte. The infusion system is configured to deliver a solution-phase therapeutic agent based on an action of the user or a control algorithm.

### CROSS REFERENCES TO RELATED APPLICATIONS

The Present application claims priority to U.S. Provisional Patent Application No. 62/823,628, filed on Mar. 25, 2019, and the Present application is a continuation-in-part application of U.S. patent application Ser. No. 16/666,259, filed on Oct. 28, 2019, which is a continuation application of U.S. patent Ser. No. 16/152,372, filed on Oct. 4, 2018, now U.S. patent Ser. No. 10/492,708 issued on Dec. 3, 2019, which is a continuation application of U.S. patent Ser. No. 15/590,105, filed on May 9, 2017, now U.S. patent Ser. No. 10/092,207, issued on Oct. 9, 2018, which claims priority to U.S. Provisional Patent Application No. 62/336,724, filed on May 15, 2016, now expired, each of which is hereby incorporated by reference in its entirety.

### STATEMENT REGARDING FEDERALLY SPONSORED RESEARCH OR DEVELOPMENT

Not Applicable

### BACKGROUND OF THE INVENTION

### Field of the Invention

The technology described herein relates to therapeutic delivery mechanisms, analyte-selective sensors and methods for configuration of the same.

### Description of the Related Art

The continuous delivery of therapeutic agents remains an important technology in modern medical devices. The most important example of such medical devices are insulin pumps, also known as continuous subcutaneous insulin infusion (CSII) systems. Insulin pumps were developed in the 1980's and commercialized in the 1990's to provide a more physiological method of insulin delivery than the infrequent injection of insulin by syringe. The importance of improved methods of insulin delivery was further recognized in the aftermath of the publication of the Diabetes Control and Complication Trial (DCTT) in 1992 which showed that intensive insulin therapy dramatically reduced the incidence and severity of long-term complications of diabetes. More recently, insulin pumps have been configured to automatically suspend insulin infusion in the event of actual or impending hypoglycemia as determined by algorithms designed to use inputs from continuous glucose monitoring systems to minimize the occurrence, severity or duration of hypoglycemia. Insulin pumps have also been configured to modulate insulin delivery continuously to maintain glucose levels at a euglycemic setpoint or within a euglycemic window (or zone) as determined by algorithms designed to use inputs from continuous glucose monitoring systems to achieve improved glycemic control. Such systems are variously described as artificial pancreas devices, automated insulin delivery systems, automated glucose control systems and closed loop systems, among other descriptions, In these scenarios, both the analyte sensing and therapeutic delivery modalities comprise two distinct and extricable devices, which are worn on the body. A major obstacle for many patients in using these technologies, however, is the use of two separate on-body devices, as shown in FIG. 2 with two devices 205 and 210 positioned on a user 215. In line with aims towards integrated sense-treat systems and the desire for miniaturized body-worn devices, the co-location of both the analyte sensing and therapeutic delivery constituents into a singular wearable device is an active area of development. With the above being said, the co-location of sensing and therapy contingents presents its own unique challenge—delivering a therapeutic agent in close proximity to an analyte sensing modality often gives rise to key technical challenges such as cross-talk, interference, contamination, and localized dilution of the analyte undergoing detection. It is for this reason that the sensing and delivery components have often been relegated to physically-separate locations on the body.

Continuous subcutaneous insulin infusion (CSII) systems are in widespread use for patients with type 1 diabetes. Increasing numbers of patients with type 2 diabetes are also using CSII systems. There are two general classes of CSII devices—one with a tube for delivering insulin and the other without a tube (or tubeless) with a small cannula protruding from the device and directly inserted into the tissue. The tubed devices consist of a programmable electromechanical pump device with an LED display and a touchpad for command entry. Tubed pumps are typically 6-8 cm long, 4-6 cm wide and 2-4 cm thick and contain a reservoir with insulin which is delivered to the body through a 24-48″ plastic tube that terminates in an infusion set with a cannula or needle that is inserted into the subcutaneous adipose tissue. The tubeless devices (sometimes referred to as patch pumps) consist of an electromechanical pump device without an LED display or a touchpad for commands, but rather with a wireless capability to a separate controller consisting of a dedicated medical device or a smart-phone running a regulated Mobile Medical Application. Patch pumps are typically 3-5 cm long, 2-4 cm wide and 2-3 cm thick and also contain a reservoir with insulin which is delivered to the body through a short 2-3 cm cannula that protrudes from the bottom of the patch pump and is also inserted into the subcutaneous adipose tissue. Current infusion systems, configured for the delivery of a solution-phase therapeutic agent (i.e. insulin) are often paired with needle- and cannula-based sensor systems configured for continuous quantification of an analyte (i.e. glucose). Although such systems operate in unison and are configured to operate in similar physiological compartments, such as the subcutaneous adipose layer of tissue, both systems are not amenable for co-location within a single body-worn device. This is due in part to the challenges associated with the insertion of two cannulae physically attached to a single integrated device. However, the primary challenge arises due to the lack of isolation while operating both systems in close proximity. Namely, undesired chemical interactions are likely to occur in scenarios of concurrent operation of an analyte sensor device and a therapeutic delivery device co-located in a given physiological compartment; among these undesired effects are cross-talk, interference, contamination, and localized dilution, which directly affect the sensor's ability to quantify the desired analyte with a specified degree of selectivity, sensitivity, stability, and response time.

The major obstacle to successful co-location of glucose sensing and insulin infusion is the deleterious effect of preservatives in current formulations of insulin on the enyzmatic properties of standard commercial glucose sensors. Ward et al. found that the phenolic preservatives present in current formulations of insulin appear to irreversibly damage the operation of glucose oxidase enzyme when sensors are operated at typical levels of bias voltage [Ward W K, Heinrich G, Breen M, Benware S, Vollum N, Morris K, Knutsen C, Kowalski J D, Campbell S, Biehler J, Vreeke M S, Vanderwerf S M, Castle J R, Cargill R S. “An Amperometric Glucose Sensor Integrated into an Insulin Delivery Cannula: In Vitro and In Vivo Evaluation” Diabetes Technol Ther. 2017 April; 19(4):226-236]. One approach to solving this problem advocated by Ward et al. is to operate the glucose sensor at a reduced bias voltage as enabled through the use of redox mediator chemistry. In glucose-oxidase based systems requiring a high applied bias voltage, the deleterious effects of the insulin preservatives require another solution to the problem of co-location of glucose sensing and insulin infusion. Our approach is based on the insight that insulin infusion by syringe or pump has a characteristic physical dispersion in the tissue that would permit simultaneous operation of a glucose sensor in the dermis and insulin infusion in the subcutaneous adipose tissue without incurring the destructive interference of the insulin preservatives on the operation of the glucose sensor. Kim et al. used high resolution x-ray imaging techniques to characterize the temporal evolution of the shape and concentration of drugs injected into porcine subcutaneous and muscle tissue [Kim H, Park H and Lee S J “Effective method for drug injection into subcutaneous tissue” Sci. Rep. 2017 Aug. 29; 7(1):9613]. They found an initial symmetric distribution followed by a subsequent asymmetric distribution in which the solution propagates faster in the horizontal direction than in the vertical distribution. Jockel and colleagues used a novel method of shock-freezing dyed insulin infused into porcine tissue with liquid nitrogen and reassembling the results of cryomicrotome slices into a three-dimensional image (Jockel J P, Roebrock P and Shergold O A “Insulin depot formation in subcutaneous tissue” J. Diabetes Sci Technol. 2013 Jan. 1; 7(1)227-237]. They found that contrary to common assumptions of a spherical depot around the tip of a syringe or insulin infusion cannula, the depot is highly asymmetric. Insulin defuses horizontally much more readily than vertically following a path of least resistance in the subcutaneous adipose tissue layer. These recent discoveries support the invention described in this application to obtain a single on-body device capable of measuring glucose in the dermis 1 mm below the surface of the skin) and simultaneously infusing insulin into the subcutaneous adipose tissue (˜10-40 mm below the surface of the skin). FIG. 11 is an illustration (not to scale) of a patient's skin 40 including epidermis 41, dermis 42 and the subcutaneous tissue 43, with an infusion system 45 configured to operate within the subcutaneous tissue 45. Graphs 1200, 1210, 1220 and 1230 of FIGS. 12A-12D below from Jockel et al shows the preferential pattern of asymmetric horizontal diffusion for different volumes of insulin injected into tissue. Jockel et al. have shown that this preferential diffusion of insulin in the horizontal direction limits the vertical extent of the insulin depot, even for large volumes of insulin, to approximately 4 mm. Hence achieving physical separation of 10 mm or more between the microneedle array in the dermis measuring glucose and the distal tip of the cannula infusing insulin in the adipose tissue is sufficient to prevent contamination of the glucose sensor and allow for physical integration of a continuous glucose monitor into an insulin delivery system.

The principles of operation of algorithms used for artificial pancreas, automated insulin delivery, automated glucose control and closed loop systems have been described in numerous review papers [Lal R A, Ekhlaspour L, Hood K, Buckingham B. “Realizing a Closed-Loop (Artificial Pancreas) System for the Treatment of Type 1 Diabetes” Endocr Rev. 2019 Dec. 1; 40(6):1521-1546; Bekiari E, Kitsios K. Thabit H, Tauschmann M, Athanasiadou E, Karagiannis T, Haidich A B, Hovorka R, Tsapas A. “Artificial pancreas treatment for outpatients with type 1 diabetes: systematic review and meta-analysis” BMJ. 2018 Apr. 18; 361:k1310; Del Favero S, Bruttomesso D, Cobelli C. “Artificial Pancreas: A Review of Fundamentals and Inpatient and Outpatient Studies” Bruttomesso D, Grassi G (eds): Technological Advances in the Treatment of Type 1 Diabetes. Front Diabetes. Basel, Karger, 2015, vol 24, pp 166-189; Peyser T, Dassau E, Breton M, Skyler J S. “The artificial pancreas: current status and future prospects in the management of diabetes” Ann NY Acad Sci. 2014 April; 1311:102-23]. Algorithms for artificial pancreas devices rely heavily on accepted principles from control theory and chemical engineering and can be divided into several categories. In the first category, algorithms for artificial pancreas devices are unihormonal or bihormonal. Unihormonal systems use insulin infusion alone to avoid hyperglycemia and maximize time in euglycemia. In unihormonal systems, hypoglycemia can be prevented or treated by suspending insulin based on actual readings of a continuous glucose monitoring systems or on predicted glucose values derived from continuous glucose monitoring data. In biohormonal systems, hyperglycemia is avoided or treated with insulin infusion as in unihormonal systems but hypoglycemia is prevented or treated by infusing glucagon which stimulates the liver to produce endogenous glucose. In the second category, optimization of insulin delivery and glycemic control can be achieved by different algorithm approaches such as Model Predictive Control (MPC), Proportional integral Derivative (PID), or Fuzzy Logic (FL). Finally, artificial pancreas can also be categorized by the degree of automation. Hybrid closed loop systems require user initiation of meal boluses based on quantitative or qualitative estimates of meal size. Full closed loop systems, by contrast, automate insulin delivery at meals and do not require user intervention for meal boluses.

Prior art solutions have largely been concerned with operating both sensing and delivery systems as extricable body-worn devices operating in the same physiological compartment, albeit spatially separated by a sufficient extent so as to avoid interactions between the two systems. Interactions can take multiple forms—cross-talk, interference, contamination, and dilution. Prior art embodiments of the analyte sensing modality include cannula-assisted, subcutaneously-implanted wire-based sensors configured to quantify an analyte using electrochemical transduction techniques. Prior art embodiments of the therapeutic delivery modality include cannula-based patch pumps and infusion sets configured to deliver a therapy to the subcutaneous adipose tissue. FIG. 1A is a prior art needle-/cannula-based analyte-selective sensor 110 with a user interface device 115 and mobile phone 105 configured for the quantification of glucose in the subcutaneous adipose tissue. FIG. 1B is a prior art needle-/cannula-based analyte-selective sensor 130 with a user interface device 125 configured for the quantification of glucose in the subcutaneous adipose tissue. FIG. 1C is a prior art needle-/cannula-based analyte-selective sensor 150 with a user interface device 145 configured for the quantification of glucose in the subcutaneous adipose tissue. More recent prior art has instructed of the co-location of both sensing and delivery modalities within a single body-worn device, albeit featuring sufficient lateral or spatial isolation between sensing and delivery contingents to minimize interactions between the two even when operating in the same physiological compartment.

Van Antwerp et al., U.S. Pat. No. 9,968,742 for a Combined sensor and infusion set using separated sites, discloses a dual insertion set for supplying a fluid to the body of a patient and for monitoring a body characteristic of the patient. Typical embodiments of the invention include a base, an infusion portion coupled to a first piercing member and a sensor portion coupled to a second piercing member. The infusion portion includes a cannula coupled to the piercing member for supplying a fluid to a placement site. The sensor portion includes a sensor coupled to and extending from the base having at least one sensor electrode formed on a substrate and is coupled to the piercing member in a manner that allows the sensor to be inserted at the placement site. The base is arranged to secure the dual insertion set to the skin of a patient. Typically the infusion portion and sensor portion piercing members are arranged such that when they are operatively coupled to the base, they are disposed in a spatial orientation designed to inhibit sensor interference that may be caused by compounds present in fluids infused through the cannula.

Curtis, U.S. Pat. No. 9,119,582 for an Integrated Analyte Sensor And Infusion Device And Methods Therefor, discloses a method and system for providing an integrated analyte monitoring system and on-body patch pump with multiple cannulas and a sensor combination is provided.

Gyrn, U.S. Patent Publication Number 20120184909, for a Delivery Device With Sensor And One Or More Cannulas, discloses a base part for a medication delivery device. The base part is during use fastened to a patient's skin and connected to a cannula part which cannula part is positioned at least partly subcutaneous. The base part is also connected to a sensor unit which can detect one or more components e.g. glucose content in the patients blood. The base part comprises fastening means (15) which fastening means (15) releasably attach the reservoir/delivery part to the base part during use and a first fluid path or means corresponding to a first fluid path from a reservoir permitting a flow of fluid between the reservoir/delivery part and the base part when the reservoir/delivery part is attached to the base part, the first fluid path comprises means (17) for interrupting the fluid flow when the detachable reservoir/delivery part is not attached to the base part (1) and opening the fluid path (19) when the delivery part is attached to the base part (1). The base part (1) also comprises a lower mounting surface (2) and one or more openings (12A, 12C) through which two or more subcutaneous units (7,70) in the form of at least one cannula and at least one sensor part or at least two cannulas extend and it comprises a second fluid path permitting a flow of fluid from the outlet of the first fluid path to an inlet of a subcutaneously positioned cannula (22, 22a, 22b) during use, and a signal path is provided from the reservoir/delivery part to a sensor contact part. The base part is characterized in that the second fluid path is in fluid connection with an end opening of a subcutaneously positioned cannula during use.

Regittnig, U.S. Patent Publication Number 20140288399, for a Medical Apparatus having Infusion And Detection Capabilities, discloses a medical apparatus for supplying a medication fluid into an organism and for detecting a substance of the organism, the medical apparatus comprising: a casing (101, 201, 301, 401, 501, 601) having a chamber (143, 243,343,443) for accommodating the medication fluid (133, 233, 333, 433) and having an opening (145, 245, 246, 345, 445) in fluid communication with the chamber; a cannula (103, 203, 303, 403) having a lumen (161, 261, 361, 461) being in fluid communication with the chamber; an insertion needle (105, 205) having a receptacle, wherein at least a portion of the insertion needle is removably arrangeable within the lumen of the cannula; a sensor system (107, 207, 307) for detecting the substance of the organism, wherein the receptacle of the insertion needle is configured to removably at least partially receive the sensor system; and a seal (153, 253, 353, 453), wherein the seal is adapted to annularly sealingly surround the insertion needle when the insertion needle is arranged in the opening, wherein the seal is adapted to annularly sealingly surround a portion (139, 239, 339, 439) of the sensor system when the insertion needle is removed from the opening and the portion (139, 239, 339, 439) of the sensor system is arranged in the opening.

Geismar et al., U.S. Patent Publication Number 2006017761 for a Dual Insertion Set, discloses a dual insertion set that includes a base (40), an infusion portion (30), a sensor portion (20), and at least one piercing member (24, 34). The base is adapted to secure the dual insertion set to the skin of a patient. The infusion portion includes a cannula (33) for supplying a fluid to a placement site. The cannula is coupled to and extends from the base and has at least one lumen with a distal end for fluid communication with the placement site. The cannula has at least one port structure formed near another end of the lumen opposite the distal end. The sensor portion includes a sensor (22) coupled to and extending from the base having at least one sensor electrode formed on a substrate. The sensor is for determining a body characteristic, e.g. the glucose level, of the patient at the placement site. The at least one piercing member is coupled to and extends from the base to facilitate insertion of the cannula and the sensor.

Yodat et al., U.S. Pat. No. 9,056,161 for a Fluid Delivery System With Electrochemical Sensing Of Analyte Concentration Levels, discloses a system and a method for delivering fluid to and sensing analyte levels in the body of the patient are disclosed. The system includes a dispensing apparatus configured to infuse fluid into the body of the patient and a sensing apparatus configured to be in communication with the dispensing apparatus and further configured to detect a level of analyte concentration in the body of the patient.

Ward et al., U.S. Patent Publication Number 20160354542, for a Measurement Of Glucose In An Insulin Delivery Catheter By Minimizing The Adverse Effects Of Insulin Preservatives, discloses a concept, and method of creating, a dual use device intended for persons who take insulin. In one embodiment, the novel device is an insulin delivery cannula, the outer wall of which contains electrodes, chemical compounds and electrical interconnects that allow continuous glucose sensing and delivery of data to a remote device. Heretofore, the main problem in attempting to sense glucose at the site of insulin delivery has been the high current resulting from oxidation by the sensor of the preservatives in the insulin formulations. One means of eliminating these interferences is to poise the indicating electrode(s) of the sensor at a bias sufficiently low to avoid the signal from oxidation of the preservatives. One way of obtaining a glucose signal at a low bias is to use an osmium-ligand-polymer complex instead of conventional hydrogen peroxide sensing. Another is to use a size exclusion filter located in line with the insulin delivery tubing in order to remove the smaller phenolic preservative molecules while allowing the larger insulin molecules to pass unimpeded. These filtration concepts can also be more broadly applied, that is, the general concept of removal of unwanted drug formulation excipients from a drug delivery system.

O'Connor et al., U.S. Patent Publication Number 20170173261, for a Wearable Automated Medication Delivery System, discloses Systems and methods for automatically delivering medication to a user. A sensor coupled to a user can collect information regarding the user. A controller can use the collected information to determine an amount of medication to provide the user. The controller can instruct a drug delivery device to dispense the medication to the user. The drug delivery device can be a wearable insulin pump that is directly coupled to the user. The controller can be part of or implemented in a cellphone. A user can be required to provide a confirmation input to allow a determined amount of insulin to be provided to the user based on detected glucose levels of the user. The sensor, controller, and drug delivery device can communicate wirelessly.

Scientific and medical researchers have identified that a reduction in the number of percutaneous devices would enable more widespread adoption of body-worn sensor and therapeutic delivery devices, thereby improving outcomes; preliminary clinical investigations have validated this approach. [Adv. Therapy 23, 725-732 (2006)] This is most evident in the field of diabetes management, where patients with diabetes may often have to wear two separate devices on their body—a continuous glucose monitor and an insulin pump—to manage their glycemic state. [U.S. Pat. No. 9,452,258]. Although the drive towards integration of both devices might seem obvious, a number of physiological and technological challenges have prevented a successful solution to this problem. The greatest obstacle to colocation of insulin infusion and glucose sensing appears to be the preservatives employed for stabilization of insulin formulations which damage the enzymes used in typical glucose sensors.

### BRIEF SUMMARY OF THE INVENTION

The invention described herein achieves the objective of placing these components within distinct physiological compartments while at the same time allowing for their integration into a single on-body device.

One aspect of the present invention is a device for the manual delivery of a therapeutic intervention in response to a physiological state of a user. The device comprises a sensor, an infusion system, a singular body-worn component and a control algorithm. The sensor is configured to penetrate the stratum corneum to access the viable epidermis or dermis and measure the presence of an analyte or plurality of analytes in a selective fashion. The infusion system is configured to deliver, in a controlled fashion, a solution-phase therapeutic agent or collection of therapeutic agents to a separate physiological compartment distinct from the viable epidermis and dermis. The singular body-worn component integrates the sensor and the infusion system. The device is configured to deliver a specified dosage of said therapeutic agent via the infusion system based on the output of said control algorithm.

Another aspect of the present invention is a device for the manual delivery of a therapeutic intervention in response to a physiological state of a user. The device comprises a sensor, an infusion system, and a singular body-worn component. The sensor is configured to penetrate the stratum corneum to access the viable epidermis or dermis and measure the presence of an analyte or plurality of analytes in a selective fashion. The infusion system is configured to deliver, in a controlled fashion, a solution-phase therapeutic agent or collection of therapeutic agents to a separate physiological compartment distinct from the viable epidermis and dermis. The singular body-worn component integrates the sensor and the infusion system. The device is configured to deliver a specified dosage of said therapeutic agent via the infusion system based on an action of a user.

Yet another aspect of the present invention is a method for the manual delivery of a therapeutic intervention in response to a physiological state of a user using a singular body-worn component. The method includes measuring the presence of an analyte or plurality of analytes in a selective fashion in the viable epidermis or dermis by means of a sensor. The method also includes presenting the measurement of an analyte or plurality of analytes to a user. The method also includes causing an infusion system to deliver a specified dosage of a solution-phase therapeutic agent or collection of therapeutic agents to a physiological compartment beneath the dermis based on an action of said user.

Yet another aspect of the present invention is a method for the manual delivery of a therapeutic intervention in response to a physiological state of a user using a singular body-worn component. The method includes measuring the presence of an analyte or plurality of analytes in a selective fashion in the viable epidermis or dermis by means of a sensor. The method also includes inputting the measurement of an analyte or plurality of analytes into a control algorithm. The method also includes causing an infusion system to deliver a specified dosage of a solution-phase therapeutic agent or collection of therapeutic agents to a physiological compartment beneath the dermis based on the output of said control algorithm.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1A is a prior art needle-/cannula-based analyte-selective sensors configured for the quantification of glucose in the subcutaneous adipose tissue.

FIG. 1B is a prior art needle-/cannula-based analyte-selective sensors configured for the quantification of glucose in the subcutaneous adipose tissue.

FIG. 1C is a prior art needle-/cannula-based analyte-selective sensors configured for the quantification of glucose in the subcutaneous adipose tissue.

FIG. 2 is a prior art embodiment of an analyte-selective sensor device (left) and an infusion system (right), both devices featuring extensive spatial separation circumvent undesired interactions.

FIG. 3 is a prior art needle-/cannula-based analyte-selective sensor (left) configured for the quantification of glucose in the subcutaneous adipose tissue and a microneedle array-based analyte-selective sensor (right) configured for the quantification of glucose in the dermis.

FIG. 4 is a pictorial representation (not to scale) of an infusion system configured to operate within the subcutaneous tissue (left) and an analyte-selective sensor configured to operate within the dermis (right), with both located in close spatial proximity.

FIG. 5A is an illustration of an integration of a microneedle array-based analyte-selective sensor into an infusion set.

FIG. 5B is a proposed integration of a microneedle array-based analyte-selective sensor into a patch pump.

FIG. 6A is a proposed integration of a microneedle array-based analyte-selective sensor into a patch pump.

FIG. 6B is a proposed integration of a microneedle array-based analyte-selective sensor into a patch pump.

FIG. 6C is an isolated view of circle 6C of FIG. 6B.

FIG. 7 is a block/process flow diagram illustrating the major method steps of the OPEN LOOP embodiment of the invention.

FIG. 8 is a block/process flow diagram illustrating the major method steps of the CLOSED LOOP embodiment of the invention.

FIG. 9 is a block/process flow diagram illustrating the inputs, outputs, and major constituents of the invention under the OPEN LOOP embodiment.

FIG. 10 is a block/process flow diagram illustrating the inputs, outputs, and major constituents of the invention under the CLOSED LOOP embodiment.

FIG. 11 is an illustration (not to scale) of an infusion system configured to operate within the subcutaneous tissue.

FIG. 12A is an illustration of a graph related to insulin depot formation in subcutaneous adipose tissue.

FIG. 12B is an illustration of a graph related to insulin depot formation in subcutaneous adipose tissue.

FIG. 12C is an illustration of a graph related to insulin depot formation in subcutaneous adipose tissue.

FIG. 12D is an illustration of a graph related to insulin depot formation in subcutaneous adipose tissue.

FIG. 13 is a top perspective view of an insulin patch pump with a fully integrated microarray sensor.

FIG. 14 is a bottom perspective view of an insulin patch pump with a fully integrated microarray sensor.

FIG. 15 is a side elevation view of an insulin patch pump with a fully integrated microarray sensor.

FIG. 16 is a bottom perspective view of an insulin patch pump with a microarray sensor connected via a connector.

FIG. 17 is an isolated view of a microarray sensor connected to a connector.

FIG. 18 is a bottom perspective view of an insulin patch pump with a recess for a microarray sensor that has been applied to a patient's skin.

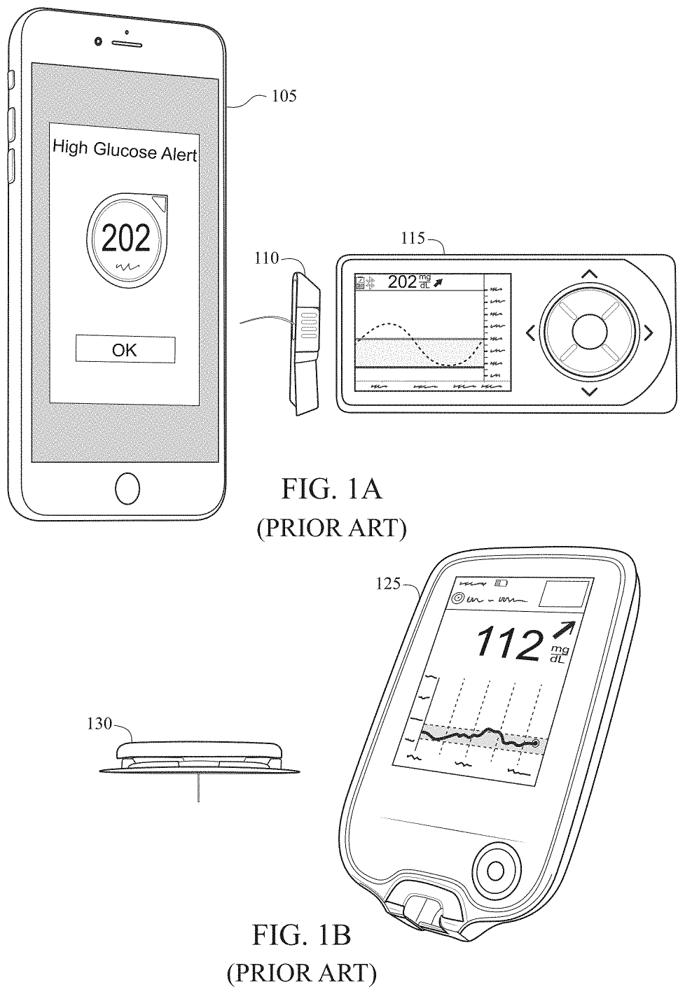

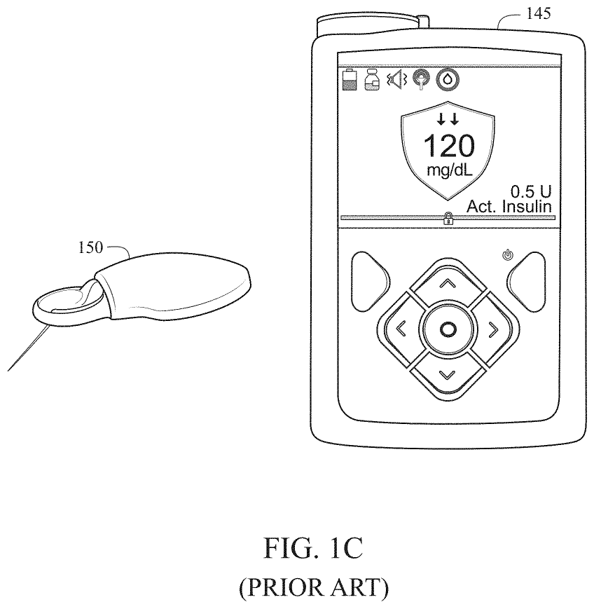

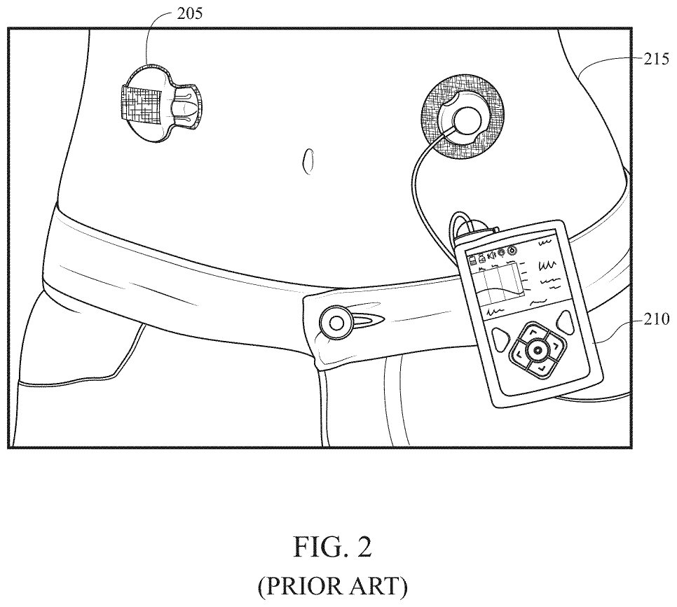

### DETAILED DESCRIPTION OF THE INVENTION

Microneedle-based analyte-selective sensors have witnessed increased development activity in recent years and represent a promising capability towards the minimally-invasive quantification of a number of relevant analytes in the physiological fluid, such as interstitial fluid, dermal interstitial fluid blood, serum, and plasma. These devices consist of an array of at least two protrusions on a substrate, each protrusion attached to the said substrate at the proximal end and extending between 200 and 2000 micrometers to a distal end. At least one said protrusion is configured to feature at least one electrode comprising a metallic, semiconductor, or polymeric material, which may be further coated with one or more polymeric membranes. In certain embodiments, a recognition element is located on the said electrode or within the said membrane to impart a selective sensing capability towards an endogenous or exogenous chemical species occupying the said physiological fluid. The said chemical species can include at least one of a biomarker, chemical, biochemical, metabolite, electrolyte, ion, hormone, neurotransmitter, vitamin, mineral, drug, therapeutic, toxin, enzyme, protein, nucleic acid, DNA, and RNA. Modes of sensing can include electrical, chemical, electrochemical (voltammetric, amperometric, potentiometric), optical, fluorometric, colormetric, absorbance, emission, conductance, impedance, resistance, capacitance. Typical modes of application include by means of a user-supplied force, a packaging wherein stored potential energy is transferred to kinetic energy upon an actuation action instigated by a user, and an applicator capable of accelerating the said microneedle-based analyte-selective sensors, causing the microneedle constituents of the array to penetrate the stratum corneum and achieve sensing within the viable epidermis or dermis to facilitate the intradermal analysis of pertinent analytes from the viable physiological medium (interstitial fluid, blood) occupying the layers of the viable epidermis and dermis. Sensing is achieved on a continuous, quasi-continuous, periodic, or single-shot fashion. The sensor device contains, in some embodiments, a wireless radio configured to relay data, measurements, or readings to connected wirelessly-enabled devices such as smartphones, smartwatches, and therapeutic delivery systems. In other embodiments, the said device contains at least one electrical contact configured to relay data, measurements, or readings to a mechanically-coupled therapeutic delivery system.

Therapeutic delivery systems are configured to infuse a therapy, drug, medication, pharmaceutical, or active ingredient into a physiological compartment of a user. These devices most commonly contain a reservoir for said therapy, a dispensing mechanism or actuator to control the quantity or dosage of said therapy, a power source (i.e. battery), and an electrical controller containing an embedded control algorithm programmed into firmware. In some preferred embodiments, these systems feature a wireless radio configured to relay data, measurements, or readings to connected wirelessly-enabled devices such as smartphones, smartwatches, and continuous analyte monitors. Embodiments of therapeutic delivery systems include skin-worn integrated patch pumps integrating both the pump and cannula for subcutaneous delivery of the therapy. In other embodiments, said therapeutic delivery systems contain a non-skin worn pump and a skin-adorned infusion set. Continuous subcutaneous insulin infusion (CSII) and automated insulin delivery (AID) systems, comprised of an insulin pump, control algorithm, and method of data interface with a continuous glucose monitor, have been at the forefront of development activities in this domain owing to the potential for closed-loop operation aimed at automating the delivery of insulin to counteract glycemic excursions and maximize the user's time in euglycemia, otherwise known as time-in-range.

Control algorithms, which are designed to modulate the delivery of a therapy via a therapeutic delivery system based on a data input provided by a continuous analyte sensor, enable the automated delivery of a therapeutic intervention, the dosage of which is controlled to counteract pathophysiological states. While basal rate delivery entails a fixed temporal rate of therapeutic delivery, the ability to measure at least one analyte provides for an effective method of feedback, hence lending itself to fully autonomous closed-loop therapy. Specifically, analyte sensors may comprise continuous glucose monitors and therapeutic delivery systems may constitute continuous subcutaneous insulin infusion (CSII) systems. Continuous glucose monitors are configured to measure glucose beneath the skin and CSII systems comprise a pump paired with an infusion set or are otherwise integrated into a skin-adhered patch (also known as a tubeless pump) and configured to deliver a prescribed dose of insulin. A control algorithm residing within the CSII, continuous glucose monitor, or wirelessly-paired device calculates the dosage of therapy required to counteract a pathophysiological state based on the readings from said continuous glucose monitor and achieve tight glycemic control, preferably within the euglycemic range (70-180 mg/dL). Said control algorithm is designed to monitor the controlled process variable (i.e. glucose level by means of the continuous glucose monitor), and compares it with the reference or set point (i.e. glucose level within euglycemic range). The difference between the actual and desired value of the process variable, called the error signal, or SP-PV error, is applied as feedback to generate a control action to bring the controlled process variable to the same value as the set point. In other terms, the primary objective of the control algorithm is the minimization of the error signal. The system can operate under closed-loop control (i.e. the control action from the controller is dependent on feedback from the process in the form of the value of the process variable) or open-loop control (i.e. the control action from the controller is independent of the process output). In various embodiments, no feedback or negative feedback may be employed. Negative feedback has the advantage that unstable processes can be stabilized, reduced sensitivity to parameter variations, and improved set point performance. In a preferred embodiment, the control algorithm resides in a memory or processor embedded within the CSII system. Typically the control algorithm (residing in a processor or memory) receives as an input glucose data manually entered by the user (such as from a finger-stick blood sample), or streamed from a continuous glucose monitor (usually wirelessly, but the processor could have a direct electrical connection co-located in a single device).

An exemplary closed-loop controller architecture is the proportional-integral-derivative (PID) controller. As with other exemplary systems, the PID controller makes extensive use of the transfer function, also known as the system function or network function, which is composed of a mathematical model (i.e. set of time- or process-dependent equations) of the relation between the input and output of the system. Another embodiment of the control algorithm can comprise of at least one of a proportional-integral-derivative, model predictive control, fuzzy logic, and safety supervision design (Ann. NY Acad. Sci. 2014 April: 1311:102-23).

The current invention teaches of devices and methods for sensing an analyte, or plurality of analytes, and delivering a concomitant therapeutic intervention, or plurality of therapeutic interventions, in distinct physiological compartments, using a single body-worn device, to avoid issues associated with cross-talk, interference, contamination, and/or dilution that arise when performing both actions in a spatial vicinity. The single body-worn device is configured to be easily applied to the skin of a wearer and engages in a sensing routine in the viable epidermis or dermis of said wearer. Delivery or infusion of a therapeutic intervention is directed at the deeper and anatomically separate and distinct subcutaneous adipose tissue layer. Embodiments can either include an open-loop system, whereby the wearer adjusts dosing of said therapeutic intervention based on levels of said analyte, or plurality of analytes, and a close-loop system, whereby a control algorithm autonomously adjusts dosing of the therapeutic intervention or plurality of therapeutic interventions.

Current needle- and cannula-based infusion systems, configured for the delivery of a solution-phase therapeutic agent (i.e. insulin) are often paired with needle- and cannula-based sensor systems configured for continuous quantification of an analyte (i.e. glucose). Although such systems operate in unison and are configured to operate in the same physiological compartment, such as the subcutaneous adipose layer of tissue, both systems are not amenable for co-location within a single body-worn device. Although this is partly due to challenges associated with the insertion of two cannulae physically attached to a single integrated device, the primary challenge arises due to the lack of isolation while operating both systems in close proximity. Namely, undesired chemical interactions are likely to occur in scenarios of concurrent operation of an analyte sensor device and a therapeutic delivery device co-habilitating a given physiological compartment; among these undesired effects are cross-talk, interference, contamination, and localized dilution, which directly affect the sensor's ability to quantify the desired analyte with a specified degree of selectivity, sensitivity, stability, and response time. As an example, insulin liquid formulations, which are often employed in insulin pumps, include m-Cresol and methyl p-hydroxybenzoate as preservative agents.¹ Although both compounds are effective in preserving the activity of insulin over extended duration of storage and in the wake of significant temperature fluctuations, these substances are electroactive and interfere with the concurrent electrochemical detection of glucose. Hence, in order to accurately measure glucose, the delivery of insulin must be spatially isolated from the sensor; this spatial isolation makes the integration of both sensing and therapeutic modalities impractical for a single body-worn device. Moreover, co-location of both the analyte sensing and therapeutic delivery contingents into a singular body-worn device has presented noteworthy challenges with respected to application due to difficulties associated with the implantation of two cannulae, either simultaneously or in progression. Taken together, there is a strong drive to integrate both the analyte sensor and therapeutic delivery modalities into a singular body-worn device for the sake of wearer convenience and simplicity. This is particularly driven by the current challenge of automated insulin delivery, which holds considerable promise for more effective management of insulin-dependent diabetes mellitus. Today, owing to current design implementations, the analyte sensing and therapeutic delivery modalities are both configured to operate in the subcutaneous adipose tissue otherwise known as the subcutis, sub-dermis, or hypodermis. If co-location of both systems into a singular body-worn device is desired, a key challenge arises—sufficient spatial separation of the analyte-sensing and therapeutic delivery modalities such that both can operate in an isolated manner (i.e. either system remaining unaffected by the routines executed at the other contingent). Moreover, albeit to a lesser extent, the co-location of both the analyte sensing and therapeutic delivery systems into a singular body-worn device with a compelling form-factor faces certain integration challenges, particularly owing to the macro-scale features of both systems and their own unique requirements for packaging, control electronics, and hardware.

Body-worn analyte sensors (such as continuous glucose monitors) are sensitive electrochemical systems that are configured to sense an analyte, or plurality of analytes, in a selective fashion with a high-degree of accuracy. In many cases, the sensor can be configured to exclude other endogenous analytes from interfering with the detection process, however, the perturbation of equilibrium conditions (such as those arising from infusion) in the vicinity of said sensor can instigate errant readings that are not reflective of the level of the analyte in situ, not to mention that a multitude of exogenous therapeutic agents can directly interfere with the quantification of said analyte. The current invention addresses the challenge of co-location of both the analyte sensing and therapeutic delivery modalities in the same on-body device in the same physiological compartment by facilitating the separation of the analyte sensing and therapeutic delivery routines in distinct physiological compartments (skin strata) that are transversely rather than laterally separated. The innovation represents an alternative approach facilitating the delivery of a therapeutic treatment without causing a subsequent and undesired response in an analyte-selective sensor operating in close proximity to the therapeutic solution; this is achieved by locating both the sensing and delivery contingents in unalike physiological compartments even in scenarios where both modalities are located in the same lateral spatial vicinity, such as within a singular body-worn device. An exemplary embodiment of the analyte sensor in this invention constitutes a microneedle or microneedle array configured to sense at least one analyte in the viable epidermal or dermal layer of the skin and a cannula-based infusion set or patch pump configured to deliver at least one of a therapeutic intervention such as a solution-phase drug, pharmacologic, biological, or medicament into subcutaneous adipose tissue layer. Transverse separation between the two contingents (the analyte sensor integrated into a patch pump or cannula-based infusion set) in various embodiments can be in the range of 2 mm to 50 mm, and most preferably from 5 mm to 25 mm. Both physiological compartments are expected to be sufficiently isolated so as to mitigate likely occurrences of cross-talk, interference, contamination, and localized dilution of the analyte undergoing detection.

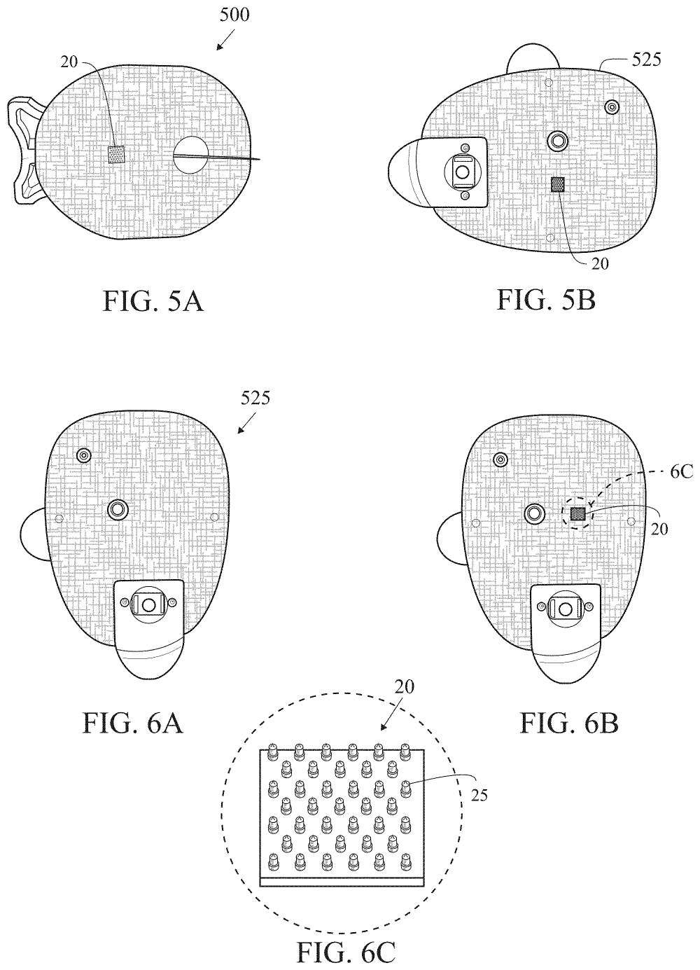

FIG. 5A is an illustration of an integration of a microneedle array-based analyte-selective sensor 20 into an infusion set 500.

FIGS. 5B, 6A and 6B illustrate the integration of a microneedle array-based analyte-selective sensor 20 into a patch pump 525. FIG. 6C illustrates the microneedle array-based analyte-selective sensor 20 and microneedles 25.

The technology disclosed herein juxtaposes the analyte sensor system and therapeutic delivery system to operate in different physiological compartments yet maintain minimum spatial separation between the two. This is achieved by dispensing the analyte sensor in the viable epidermis or dermis of a wearer, whereby the system is configured to quantify an analyte, or plurality of analytes, residing therein. Conversely, the therapeutic delivery system is dispensed in the subcutaneous region. Transverse separation of both the sensing and delivery modalities, confining the sensing routine to the viable epidermis or dermis and delivery routine to the subcutaneous adipose tissue, enables the isolation of both routines, thus mitigating likely occurrences of cross-talk, interference, contamination, and localized dilution of the analyte undergoing detection should both be co-located in a given physiological compartment. In preferential embodiments of this invention, the system can function under an open-loop paradigm whereby therapy is instigated by a user and guided by measurements from said sensor. Alternatively, the system can feature a control algorithm to autonomously deliver a therapeutic intervention in response to a sensor reading or plurality of readings. It is expected that this paradigm will have profound implications for diabetes management and, in particular, those who are undergoing intensive insulin therapy.

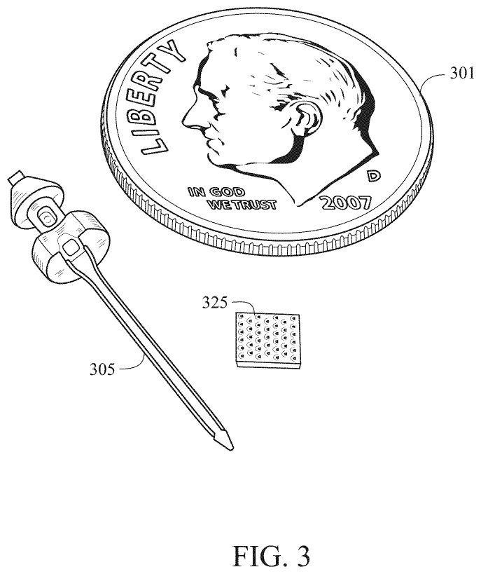

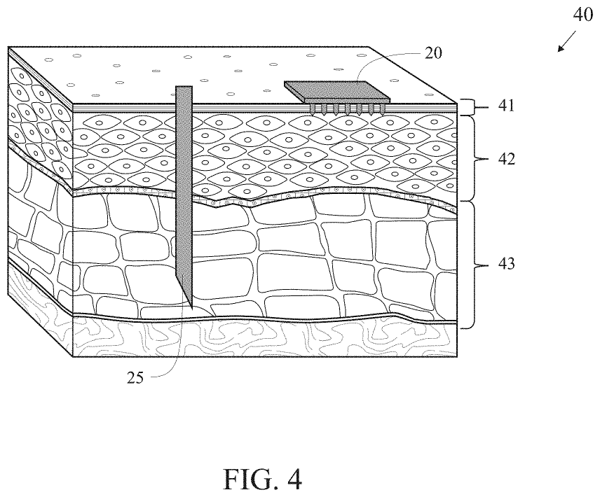

An open loop embodiment of the present invention comprises a system integrating a sensor and an infusion sub-system and requires an action of a user to instigate the delivery of a therapeutic intervention. The system is preferably a body-worn device capable of incorporating both the sensor and the infusion sub-system to deliver a therapeutic agent in a physically-distinct compartment from the region in which the analyte is detected. The sensor is preferably a plurality of microneedles, possessing vertical extent between 200 and 2000 μm, configured to selectively quantify the levels of at least one analyte located within the viable epidermis or dermis. FIG. 3 illustrates the microneedle array sensor 325 in relation to a dime 301 and needle 305. The sensor is designed to measure the analyte in one distinct layer of the skin, for example, the viable epidermis or dermis. The infusion sub-system is designed to deliver the therapeutic agent to a different and physically distinct layer of the skin, for example the subcutaneous adipose tissue. The infusion sub-system is preferably a fluid delivery apparatus configured to provide infusion of a solution-phase therapeutic agent into the subcutaneous adipose layer, circulatory system (venous, arterial, or capillary), or musculature via intravenous line, hypodermic needle, infusion cannula or oral delivery route. The therapeutic agent is preferably a solution-phase drug, pharmacologic, biological, or medicament. The sensor may be incorporated onto the bottom of an insulin infusion cannula set adhered to the skin with medical adhesive and attached to the insulin pump by a hollow plastic tube. Alternatively, the sensor may be incorporated onto the bottom of an insulin patch pump adhered to the skin with medical adhesive.

A closed loop embodiment of the present invention comprises a system integrating a sensor and an infusion sub-system and employs a control algorithm to instigate the delivery of a therapeutic intervention. The system is preferably a body-worn device, with a control algorithm, capable of incorporating both the sensor and the infusion sub-system to deliver a therapeutic agent in a physically-distinct compartment from the region in which the analyte is detected. The sensor is preferably a plurality of microneedles, possessing vertical extent between 200 and 2000 μm, configured to selectively quantify the levels of at least one analyte located within the viable epidermis or dermis. The sensor is designed to measure the analyte in one distinct layer of the skin, for example, the viable epidermis or dermis. The infusion sub-system is designed to deliver the therapeutic agent to a different and physically distinct layer of the skin, for example the subcutaneous adipose tissue. The infusion sub-system is preferably a fluid delivery apparatus configured to provide infusion of a solution-phase therapeutic agent into the subcutaneous adipose layer, circulatory system (venous, arterial, or capillary), or musculature via intravenous line, hypodermic needle, infusion cannula or oral delivery route. The therapeutic agent is preferably a solution-phase drug, pharmacologic, biological, or medicament. The sensor may be incorporated onto the bottom of an insulin infusion cannula set adhered to the skin with medical adhesive and attached to the insulin pump by a hollow plastic tube. Alternatively, the sensor may be incorporated onto the bottom of an insulin patch pump adhered to the skin with medical adhesive. Therapy is defined as the dose profile of the therapeutic agent in response to the measurement of the analyte, which may be controlled by an algorithm. The control algorithm is preferably a software or firmware routine employing one or more mathematical transformations to control dosing of a therapeutic agent, either by means of controlling the quantity delivered, duration of delivery, and/or frequency of delivery, based on input from a user or from measurements recorded by a microneedle array analyte-selective sensor. The mathematical transformation can employ additional inputs, either provided by a user or integrated autonomously from elsewhere.

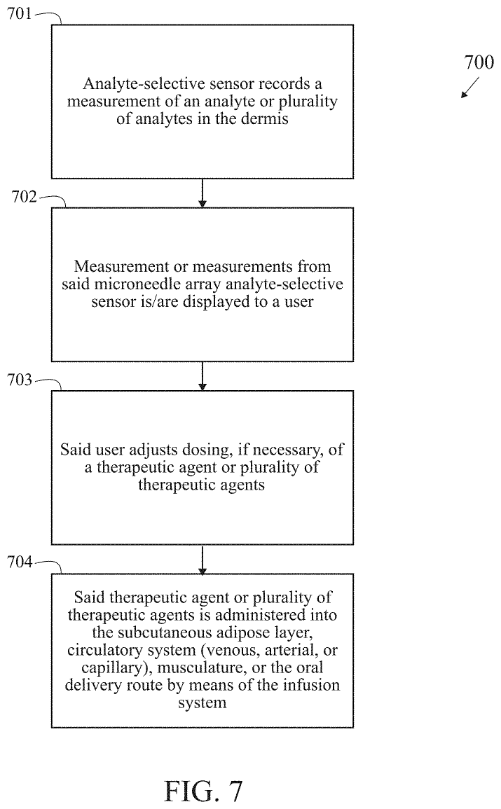

FIG. 7 is a block/process flow diagram illustrating the major method steps of the OPEN LOOP embodiment of the invention. The method 700 for performing the open loop embodiment begins at block 701 with a microneedle array analyte-selective sensor recording a measurement of an analyte or plurality of analytes in the viable epidermis or dermis. Circulating levels of an analyte within the viable epidermis or dermis is quantified by means of the sensor. Next, at block 702, a measurement or measurements from the microneedle array analyte-selective sensor is displayed to a user. The user receives a reading of the circulating level of an analyte or plurality of analytes on a display or interface. Alternatively, user receives notification that the circulating level of an analyte or plurality of analytes extends beyond a pre-defined criteria or range of values. Next, at block 703, the user adjusts dosing, if necessary, of a therapeutic agent or plurality of therapeutic agents. The user manipulates a quantity, duration, or frequency of infusion of the therapy based on measurement of analyte or plurality of analytes tendered by the sensor. Next, at block 704, the therapeutic agent or plurality of therapeutic agents is administered into the subcutaneous adipose layer, circulatory system (venous, arterial, or capillary), musculature or oral delivery route by means of the therapeutic delivery mechanism. The therapy is delivered to the user via the infusion sub-system and is based on the user's determination of dosage given measurement or measurements from the sensor.

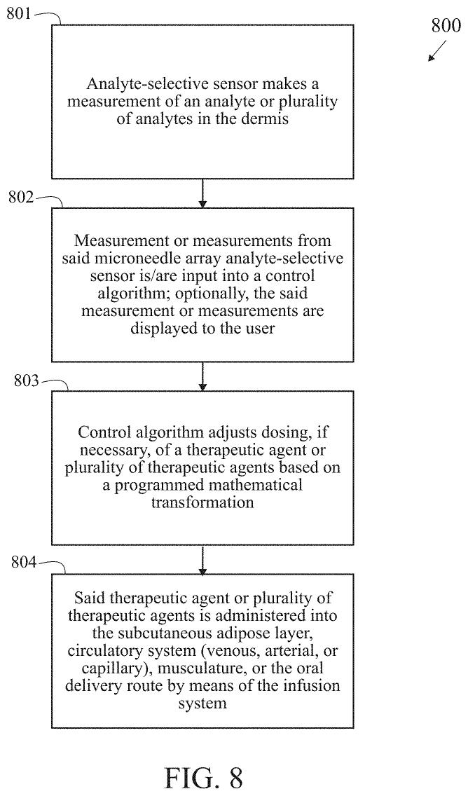

FIG. 8 is a block/process flow diagram illustrating the major method steps of the CLOSED LOOP embodiment of the invention. The method 800 for performing the closed loop embodiment begins at block 801 with a microneedle array analyte-selective sensor recording a measurement of an analyte or plurality of analytes in the viable epidermis or dermis. Circulating levels of an analyte within the viable epidermis or dermis is quantified by means of the sensor. Next, at block 802, a measurement or measurements from the microneedle array analyte-selective sensor is input into a control algorithm; optionally, the measurement or measurements are displayed to the user. Current and, optionally, past stored measurements are employed as input or inputs into the algorithm. Alternatively, the user also receives a reading of the circulating level of an analyte or plurality of analytes on a display or interface. Alternatively, the user receives notification that the circulating level of an analyte or plurality of analytes extends beyond a pre-defined criteria or range of values. Next, at block 803, the control algorithm adjusts dosing, if necessary, of a therapeutic agent or plurality of therapeutic agents based on a programmed mathematical transformation. The algorithm autonomously manipulates a quantity, duration, or frequency of infusion of the therapy based on measurement of analyte or plurality of analytes tendered by the sensor. Next, at block 804, the therapeutic agent or plurality of therapeutic agents is administered into the subcutaneous adipose layer, circulatory system (venous, arterial, or capillary), musculature or oral delivery route by means of the therapeutic delivery mechanism. The therapy is delivered to the user via the infusion sub-system and is based on the determination of dosage given output of the algorithm.

The input of circulating levels of an analyte or plurality of analytes within the viable epidermis or dermis is an endogenous or exogenous biochemical agent, metabolite, drug, pharmacologic, biological, or medicament in the viable epidermis or dermis, indicative of a particular physiological or metabolic state.

The output is an administration of a therapeutic agent or plurality of therapeutic agents into the circulatory system (venous, arterial, or capillary), musculature or oral delivery route. A measurement tendered by the sensor is employed to instigate the release of the therapy by means of the infusion sub-system. In the open loop embodiment, the delivery of the therapy is controlled by a user. In the closed loop embodiment, the algorithm is employed to control the dose, duration, and frequency of the therapy.

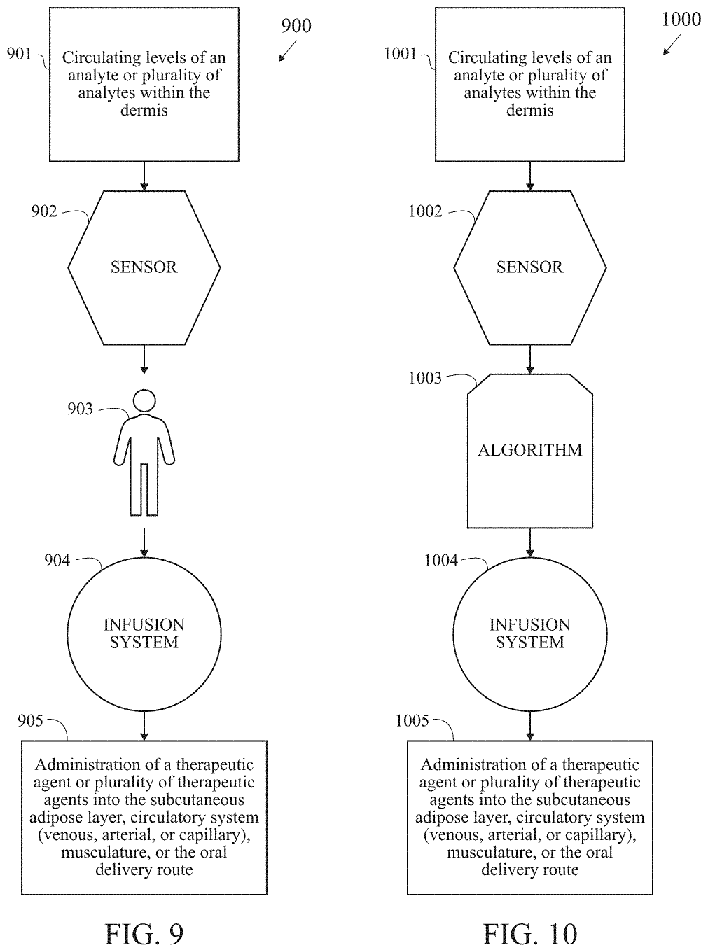

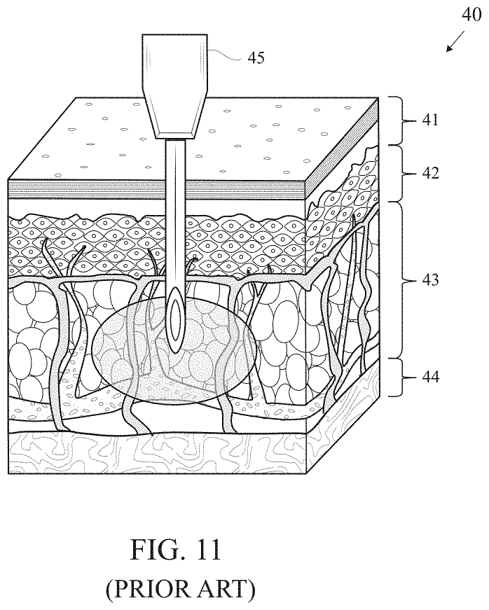

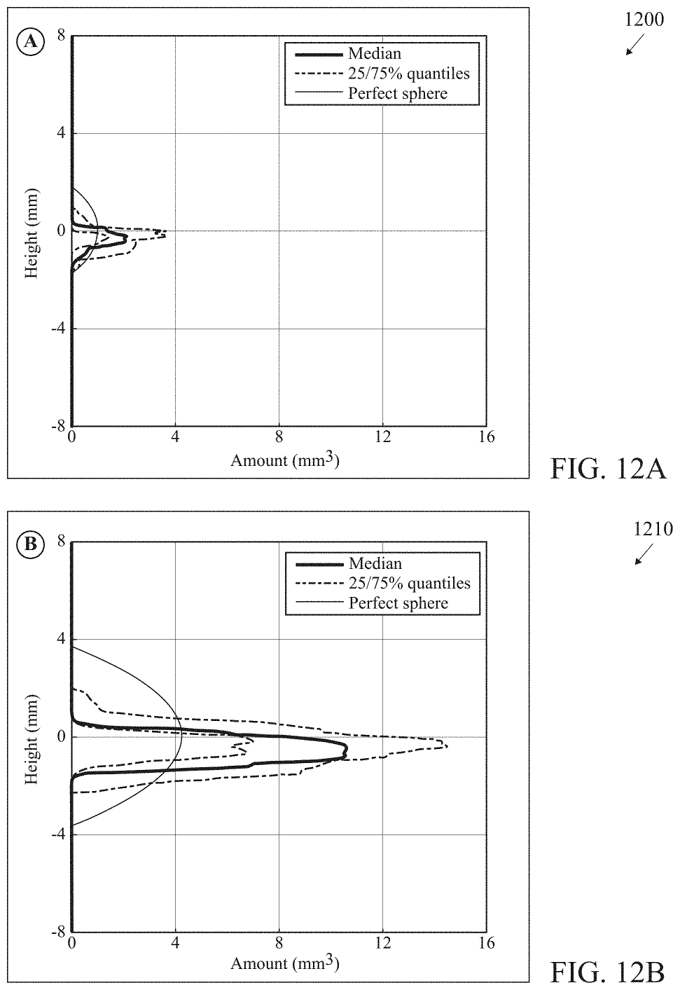

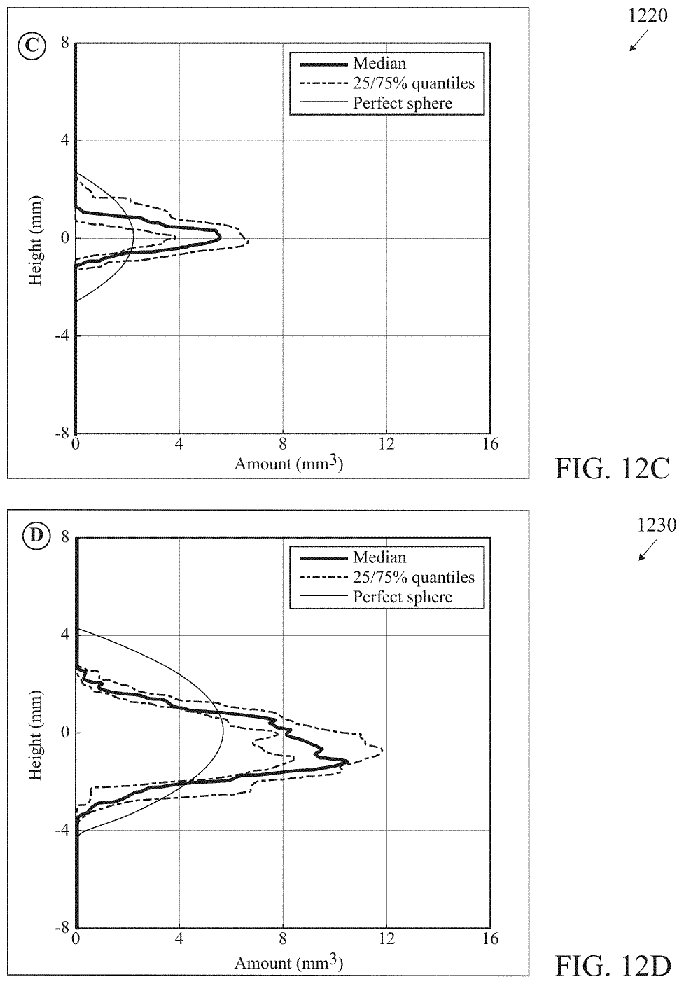

FIG. 9 is a block/process flow diagram 900 illustrating the inputs, outputs, and major constituents of the invention under the OPEN LOOP embodiment. At block 901, circulating levels of an analyte or an analytes are within the dermis. At block 902, a sensor measures the analytes. At block 903, the user adjusts dosing, if necessary, of a therapeutic agent or plurality of therapeutic agents. The user 903 manipulates a quantity, duration, or frequency of infusion of the therapy 904 based on measurement of analyte or plurality of analytes tendered by the sensor. At block 905, the therapeutic agent or plurality of therapeutic agents is administered into the subcutaneous adipose layer, circulatory system (venous, arterial, or capillary) musculature or oral delivery route by means of the therapeutic delivery mechanism. The therapy is delivered to the user via the infusion sub-system and is based on the user's determination of dosage given measurement or measurements from the sensor.

FIG. 10 is a block/process flow diagram 1000 illustrating the inputs, outputs, and major constituents of the invention under the CLOSED LOOP embodiment. At block 1001, circulating levels of an analyte or an analytes are within the dermis. At block 1002, a sensor measures the analytes. The control algorithm 1003 adjusts dosing, if necessary, of a therapeutic agent or plurality of therapeutic agents based on a programmed mathematical transformation. The algorithm autonomously manipulates a quantity, duration, or frequency of infusion of the therapy 1004 based on measurement of analyte or plurality of analytes tendered by the sensor. Next, at block 1005, the therapeutic agent or plurality of therapeutic agents is administered into the subcutaneous adipose layer, circulatory system (venous, arterial, or capillary), musculature or oral delivery route by means of the therapeutic delivery mechanism. The therapy is delivered to the user via the infusion sub-system and is based on the determination of dosage given output of the algorithm

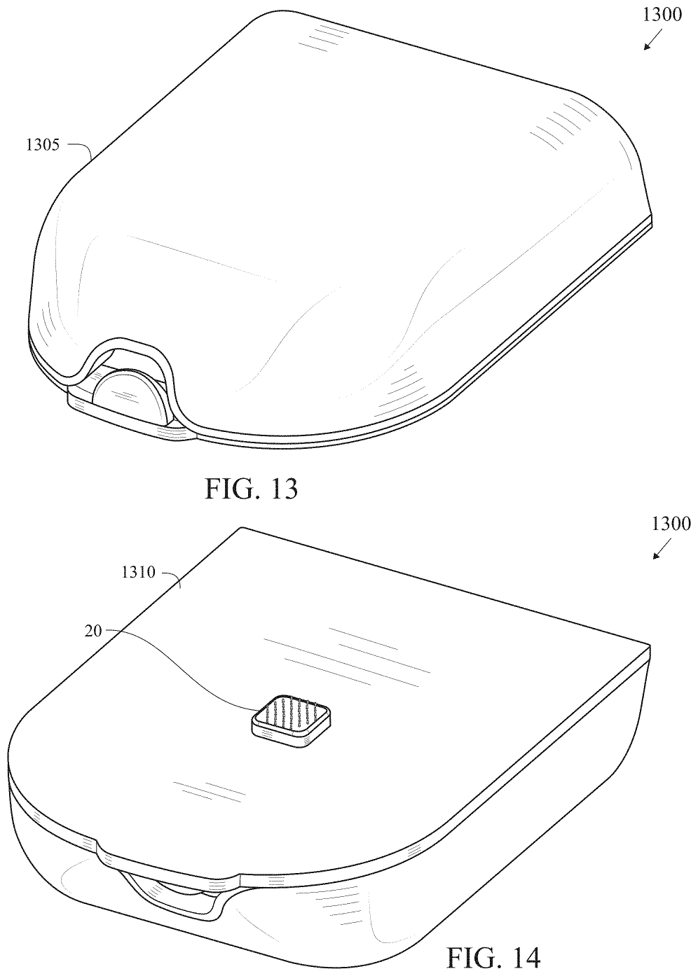

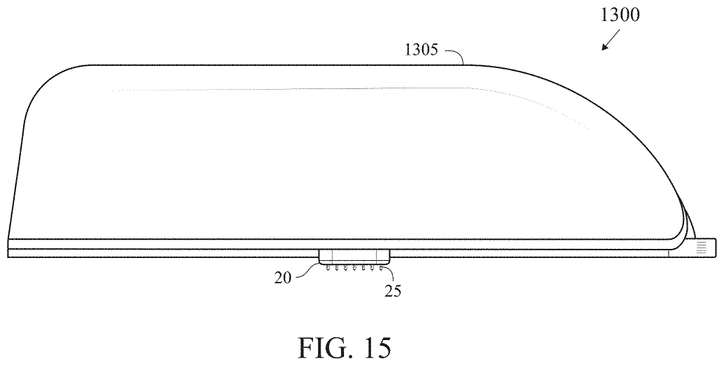

FIGS. 13-15 illustrate an insulin patch pump 1300 having a body 1305 with a fully integrated microarray sensor 20 in a bottom surface 1310.

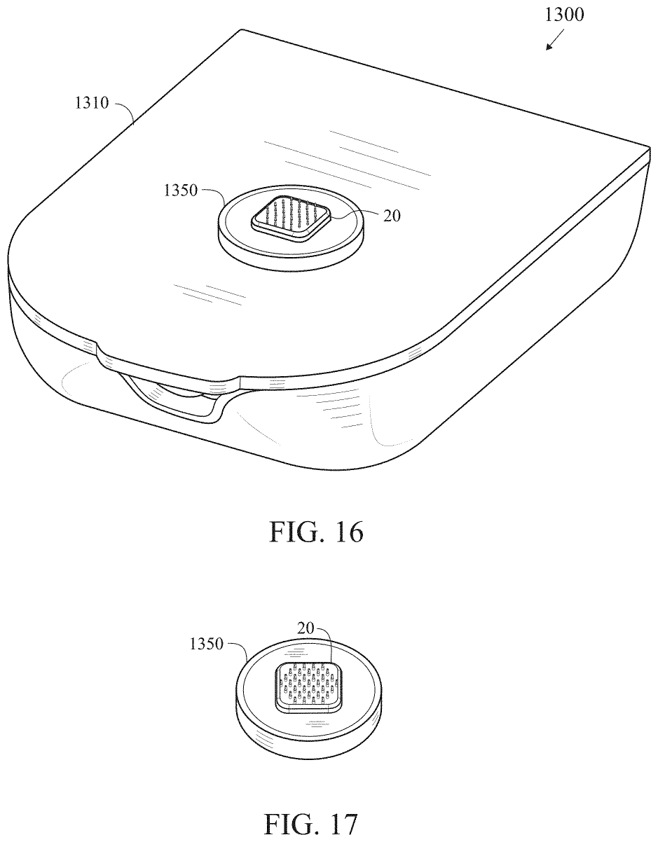

FIGS. 16-17 illustrate an alternative embodiment with an insulin patch pump 1300 with a microarray sensor 20 connected via a connector 1350 on a bottom surface 1310 of the patch pump 1300.

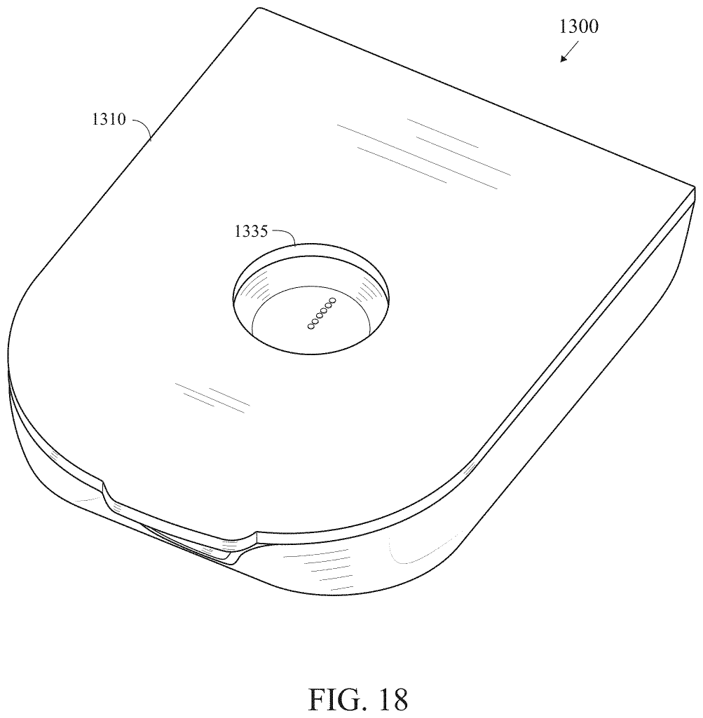

FIG. 18 illustrates an insulin patch pump 1300 with a recess 1335 in a bottom surface 1310 of the patch pump 1300 for positioning over a microarray sensor (not shown) that has already been applied to a patient's skin

McCanna et al., U.S. patent application Ser. No. 14/843,926, filed on Sep. 2, 2015, for a Miniaturized Sub-Nanoampere Sensitivity Low-Noise Potentiostat System is hereby incorporated by reference in its entirety.

Windmiller et al., U.S. patent application Ser. No. 14/955,850, filed on Dec. 1, 2015, for a Method And Apparatus For Determining Body Fluid Loss is hereby incorporated by reference in its entirety.

Windmiller, U.S. patent application Ser. No. 15/177,289, filed on Jun. 8, 2016, for a Methods And Apparatus For Interfacing A Microneedle-Based Electrochemical Biosensor With An External Wireless Readout Device is hereby incorporated by reference in its entirety.

Wang et al., U.S. Patent Publication Number 20140336487 for a Microneedle Arrays For Biosensing And Drug Delivery is hereby incorporated by reference in its entirety.

Windmiller, U.S. patent application Ser. No. 15/590,105 for a Tissue-Penetrating Electrochemical Sensor Featuring A Co Electrodeposited Thin Film Comprised Of A Polymer And Bio-Recognition Element is hereby incorporated by reference in its entirety.

Windmiller, et al., U.S. patent application Ser. No. 15/913,709, filed on Mar. 6, 2018, for Methods For Achieving An Isolated Electrical Interface Between An Anterior Surface Of A Microneedle Structure And A Posterior Surface Of A Support Structure is hereby incorporated by reference in its entirety.

PCT Application Number PCT/US17/55314 for an Electro Deposited Conducting Polymers For The Realization Of Solid-State Reference Electrodes For Use In Intracutaneous And Subcutaneous Analyte-selective Sensors is hereby incorporated by reference in its entirety.

Windmiller et al., U.S. patent application Ser. No. 15/961,793, filed on Apr. 24, 2018, for Heterogeneous Integration Of Silicon-Fabricated Solid Microneedle Sensors And CMOS Circuitry is hereby incorporated by reference in its entirety.

Windmiller et al., U.S. patent application Ser. No. 16/051,398, filed on Jul. 13, 2018, for Method And System For Confirmation Of Microneedle-Based Analyte-Selective Sensor Insertion Into Viable Tissue Via Electrical Interrogation is hereby incorporated by reference in its entirety.

Windmiller et al., U.S. patent application Ser. No. 16/701,784, filed on Dec. 3, 2019, for Devices And Methods For The Generation Of Alerts Due To Rising Levels of Circulating Ketone Bodies In Physiological Fluids is hereby incorporated by reference in its entirety.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

We claim as our invention the following:

1. A device for the manual delivery of a therapeutic intervention in response to a physiological state of a user, said device comprising:
a sensor configured to penetrate the stratum corneum to access the viable epidermis or dermis and measure the presence of an analyte or plurality of analytes in a selective fashion;
an infusion component configured to deliver, in a predetermined amount, a solution-phase therapeutic agent or a plurality of therapeutic agents to a separate physiological compartment distinct from the viable epidermis and dermis; and
a singular body-worn component for integrating said sensor and said infusion component, wherein said singular body-worn component is configured to deliver a specified dosage of said therapeutic agent via the infusion component based on an action of a user informed by a measurement from said sensor.

2. The device of claim 1, wherein said therapeutic intervention includes at least one of a solution-phase drug, pharmacologic, biological, or a medicament.

3. The device of claim 1, wherein said physiological state includes at least one of an acute metabolic condition, a chronic metabolic condition, a disease, an injury, an illness, a disorder, or an infection.

4. The device of claim 1, wherein said sensor is an electrochemical sensor, a chemical sensor, an electrical sensor, a potentiometric sensor, an amperometric sensor, a voltammetric sensor, a galvanometric sensor, an impedimetric sensor, a conductometric sensor, or a biosensor.

5. The device of claim 1, wherein said analyte or plurality of analytes includes at least one of glucose, lactate, a ketone body, uric acid, ascorbic acid, alcohol, glutathione, hydrogen peroxide, a metabolite, an electrolyte, an ion, a drug, a pharmacologic, a biological, or a medicament.

6. The device of claim 1, wherein said infusion component comprises a fluid delivery apparatus configured to provide infusion via a macroneedle, hypodermic needle, cannula, catheter, or oral delivery route.

7. The device of claim 1, wherein said singular body-worn component comprises a skin patch, a dermal patch, an adhesive patch, an infusion set, a patch pump, or an automated therapeutic delivery system.

8. The device of claim 1, wherein said specified dosage comprises a quantity of therapeutic agent, concentration of therapeutic agent, volume of therapeutic agent, duration of delivery of therapeutic agent, or frequency of delivery of therapeutic agent.

9. A device for the automated delivery of a therapeutic intervention in response to a physiological state of a user, said device comprising:
a sensor configured to penetrate the stratum corneum to access the viable epidermis or dermis and measure the presence of an analyte or plurality of analytes in a selective fashion;
an infusion component configured to deliver, in a predetermined amount, a solution-phase therapeutic agent or a plurality of therapeutic agents to a separate physiological compartment distinct from the viable epidermis and dermis;
a singular body-worn component for integrating said sensor and said infusion component; and
a control algorithm;
wherein said singular body-worn component is configured to deliver a specified dosage of said therapeutic agent via the infusion system based on the output of said control algorithm.

10. The device of claim 9, wherein said control algorithm comprises a software or firmware routine employing at least one mathematical transformation.

11. The device of claim 10, wherein the input of said mathematical transformation is the measurement of said analyte or plurality of analytes.

12. The device of claim 9, wherein the said output of said control algorithm is said specified dosage of said therapeutic agent.

13. A method for the manual delivery of a therapeutic intervention in response to a physiological state of a user using a singular body-worn component, said method comprising:
measuring the presence of an analyte or plurality of analytes in a selective fashion in the viable epidermis or dermis by means of a sensor;
presenting the measurement of an analyte or plurality of analytes to a user; and
causing an infusion component to deliver a specified dosage of a solution-phase therapeutic agent or collection of therapeutic agents to a physiological compartment beneath the dermis based on an action of said user or based on the output of said control algorithm.

14. The method of claim 13, wherein said physiological state includes at least one of an acute metabolic condition, a chronic metabolic condition, a disease, an injury, an illness, a disorder, and an infection.

15. The method of claim 13, wherein said analyte or plurality of analytes includes at least one of glucose, lactate, a ketone body, uric acid, ascorbic acid, alcohol, glutathione, hydrogen peroxide, a metabolite, an electrolyte, an ion, a drug, a pharmacologic, a biological, or a medicament.

16. The method of claim 13, wherein said sensor is an electrochemical sensor, a chemical sensor, an electrical sensor, a potentiometric sensor, an amperometric sensor, a voltammetric sensor, a galvanometric sensor, an impedimetric sensor, a conductometric sensor, or a biosensor.

17. The method of claim 13, wherein said control algorithm comprises a software or firmware routine employing at least one mathematical transformation.

18. The method of claim 13, wherein said physiological compartment beneath the dermis comprise the subcutaneous adipose tissue (the subdermis, the subcutis, the hypodermis), or musculature.

19. The method of claim 13, wherein said specified dosage comprises a quantity of therapeutic agent, concentration of therapeutic agent, volume of therapeutic agent, duration of delivery of therapeutic agent, or frequency of delivery of therapeutic agent.

20. The method of claim 13, wherein said output of said control algorithm is a signal, stimulus, or actuation indicative of a dosing of said therapeutic agent.
